(19) United States
(12) Patent Application Publication
(10) Pub. No.: US 2025/0134809 A1
(43) Pub. Date: May 1, 2025

(54) A PHYTOACTIVE ENCAPSULATED FORMULATION FOR NEURODEGENERATIVE DISEASES

(71) Applicant: Gidaa Life Sciences Pvt. Ltd., Bangalore (IN)

(72) Inventor: G. Mohamed Arif, Chennai (IN)

(21) Appl. No.: 18/837,866

(22) PCT Filed: Feb. 6, 2023
(86) PCT No.: PCT/IN2023/050114

§ 371 Date: Aug. 12, 2024

(30) Foreign Application Priority Data
Feb. 28, 2022 (IN) 202241010878

(51) Int. Cl. A61K 9/107; A61K 31/12; A61K 31/365; A61K 31/4525; A61K 36/67

(52) U.S. Cl. CPC A61K 9/1075; A61K 31/12; A61K 31/365; A61K 31/4525; A61K 36/67

(57) ABSTRACT

The present invention relates to a preparation of phospholipid encapsulated formulation (107) of curcuminoids (101) and andrographolides (102) derivate to arrest the progression of degeneration of neurons and to reduce the free radical accumulation thereby preventing the formation of Amyloid beta and Tau tangles formation. The formulation may contain either one of the curcuminoids or in combination or their analogues or their derivatives. Both curcuminoids and andrographolides either one of Curcuminoids or in combination of them can be used. Both the compounds have undergone a special process of lipid treatment and encapsulation (104, 105) for better absorption and enhanced bio-availability for better therapeutic efficacy. Piperine (103) (Extracted from Black pepper) or Black pepper itself can also be added to increase the absorption and to increase its bio availability.

### FIELD OF INVENTION

The present invention relates to the field of herbal and natural derived medicines. More particularly the invention relates to the derivatives of Curcuminoids and Andrographolides in preparing phospholipid encapsulated formulations for the prevention and treatment of neurodegenerative and other diseases.

### BACKGROUND OF THE INVENTION

Alzheimer's disease (AD) is a major health problem worldwide, with no effective treatment approach. Curcumin is the main ingredient of turmeric traditionally used in Asian medicine. Several experimental studies have indicated the protective effect of curcumin and its novel formulations in AD. Curcumin has antioxidant, anti-inflammatory and neurotrophic activities, proposing a strong potential to prevent neurodegenerative diseases.

Alzheimer's disease is an irreversible, progressive brain disorder that slowly destroys memory and thinking skills and, eventually, the ability to carry out the simplest tasks. It is the most common cause of dementia in older adults. While dementia is more common as people grow older, it is not a normal part of aging.

In most people with Alzheimer's, symptoms first appear later in life. Estimates vary, but experts suggest that more than 6 million Americans, most of them age 65 or older, may have dementia caused by Alzheimer's. Alzheimer's-related deaths in India increased 5 times in 30 years. According to the Indian government's population projections, by 2031, the country's elderly population will touch 194 million.

Alzheimer's disease is a progressive brain disease. It is characterized by changes in the brain including amyloid plaques and neurofibrillary, or tau, tangles—that result in loss of neurons and their connections. These and other changes affect a person's ability to remember and think and, eventually, to live independently.

Several medications are now available to treat symptoms of Alzheimer's. Donepezil, rivastigmine, and galantamine are used to treat the symptoms of mild to moderate Alzheimer's. Donepezil, Memantine, the Rivastigmine patch, and a combination medication of memantine and Donepezil are used to treat moderate to severe Alzheimer's symptoms.

All of these drugs work by regulating neurotransmitters, the chemicals that transmit messages between neurons. They may help reduce symptoms and help with certain behavioral problems. However, these drugs don't change the underlying disease process. They are effective for some but not all people and may help only for a limited time. In the long run and in later stages of the disease, antipsychotic and antidepressant medications are used to reduce aggression or depression. Recently USFDA has approved a monoclonal antibody treatment Aducanumab, a disease modifying immunotherapy. This has to be administered intravenously under medical supervision. This helps to remove the abnormal beta amyloid and the plaques in the brain. The side effects of aducanumab are fluid buildup in the brain, head ache, drowsiness and bleeding in the brain.

Currently there is no cure that exists for Alzheimer's disease but the availability of Medicines today can help to reduce symptoms but does not give a complete cure.

They are mainly, Acetyl Cholinesterase inhibitors like, Donepezil, Galantamine and Rivastigmine. These can increase the levels of Acetyl Choline, a neuro transmitter in the brain. Glutamate regulators like, Memantine, are another class of regulators which blocks the effect of excessive amount of glutamate which reduces the symptoms of neurogenerative diseases. These medications help to some extent to relieve symptoms and with side effects like nausea, vomiting, confusion and muscle weakness.

Donepezil is widely used for symptomatic relief for a short span of time and effective only in a small group of patients. Also, Donepezil is also associated with many undesirable side effects like, diarrhea, muscle cramps, trouble in sleeping and vomiting which are common and less common side effects like constipation, dizziness, drowsiness, fainting, mental depression, headache unusual bleeding or bruising, weight loss and bloating, blurred vision, cataract, chills, confusion, high or low blood pressure, irregular heartbeat, loss of bowel and bladder control etc. There are also symptoms of overdose like, convulsions (seizures), increased sweating, low B.P. and slow heartbeat etc.

Aluminium Chloride (AlCl3) is a potent neurotoxin and its enhancement in the brain tissues is related with the cognitive deficiency and dementia. Furthermore, AlCl3 interrupts the cholinergic neurotransmission, by depleting Acetyl Choline, the neurotransmitter and there is an increase in the AChE (Acetyl cholinesterase-which breaks down acetylcholine) activity in the brain of dementia patients. AlCl3 further leads to functional impairments of memory loss and cognitive decline by progressive accumulation of amyloid-beta “plaques”, which are cleaved protein fragments from Amyloid Precursor Proteins (APP), and neurofibrillary tangles composed of microtubule associated hyperphosphorylated tau proteins with the activation of secretases.

Important aspect of all these treatment options currently available is that they only address the symptoms and they do not address the cause and cannot arrest the progressing neuro-degeneration, hence no treatment is available.

The below patents discuss different medicinal treatments and formulations for treating neuro degenerative diseases like Alzheimer's, etc.:

The patent U.S. Pat. No. 11,179,375B2 (Methods and drug products for treating Alzheimer's disease) relates to the diagnosis and treatment of Alzheimer's disease by identifying genetic markers in the test blood sample of the patients and administering low dose pioglitazone to the patients determined to be at increased risk in developing cognitive impairment of the Alzheimer's type.

The patent KR101141439B1 (Composition comprising the extract of Zingiberis Rhizoma Crudus or Zingiberis Siccatum Rhizoma for prevention and treatment of memory and cognitive impairments involved disorder) relates to a composition for the prevention and treatment of forgetfulness and memory disorders-related diseases containing ginger or health extract as an active ingredient.

Though both patents discuss about neuro degenerative treatments, their methods are different and in the above stated scenario of available treatment options, the present invention of phyto-actives based formulation offers not only symptomatic relief but also arrests the disease progression and has potential to offer a cure for the disease. Both the ingredients (Curcuminoids and Andrographolides) being non water soluble they are poorly absorbed and have very low bioavailability. Therefore, both these ingredients undergo special process of lipid treatment and encapsulation to overcome the poor bioavailability problem and to have enhanced absorption.

Curcuminoids: Curcuminoids active principle is derived from rhizomes of plant Curcuma longa, which is a member of the family Zingiberaceae and it is commonly known as Turmeric. Curcuminoids are natural polyphenol compounds. Among them, curcumin, with bright yellow color, is the principal composition. It has long been used as food, coloring agent, and traditional medicine. A number of preclinical studies have demonstrated anticancer effects of curcumin and other curcuminoids in various types of tumors. Comprehensive research over the last century has revealed several important functions of curcuminoids. Various preclinical and animal studies suggest that curcuminoids have biological activity as an antioxidant, neuroprotective, antitumor, anti-inflammatory, anti-acidogenic, radio protective and Anti-arthritic.

Curcuminoids are a mixture of Curcumin which is chemically known as Diferuloylmethane and two of its derivates namely, Demethoxy Curcumin and Bis-demethoxy curcumin, their molecular formulae being, C21H20O6, C20H18O5 and C19H16O4 respectively. The pharmacological activity has been attributed mainly to these Curcuminoids and the three main curcuminoids are as follows

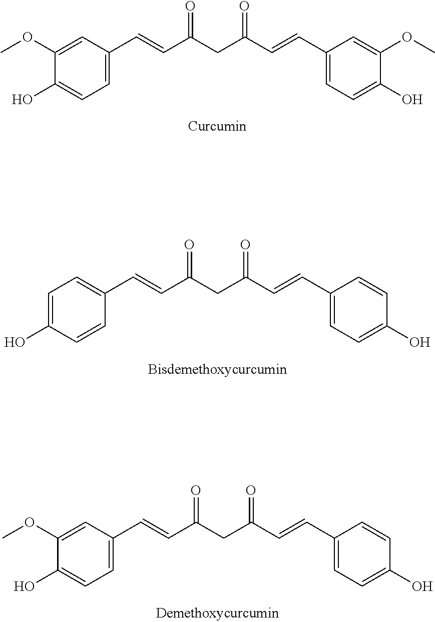

Andrographolide is derived from the plant Andrographis paniculata, commonly known as Creat or Green chireta a member of Acanthaceae family. Andrographolide is labdane diterpenoid that has been isolated from the stem and leaves of the plant. Andrographolide is an extremely bitter substance and is studied for its effects on immunomodulation, cell signaling and stroke. It also been widely used in lung infection and treatment. Its biological properties include Anti-oxidant, Anti-inflammatory and Anti-cancer properties.

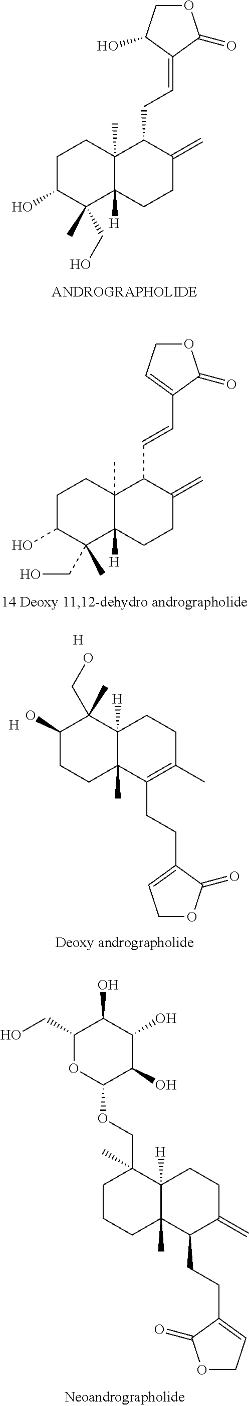

To overcome the above-mentioned draw backs, there is a need of an invention that addresses the degeneration of neurons that arrests the progression of neuro degeneration as well as to help people to maintain the mental function for treating the underlying disease process, and managing behavioral symptoms.

### OBJECTIVE OF THE INVENTION

The primary objective of the present invention is to arrest the progression of degeneration of neurons.

Another objective of the present invention is to reduce and prevent the free radical accumulation, thereby prevent the formation of Amyloid beta and Tau tangles.

Further objective of the present invention is to restore circulation to the brain and therefore it has the potential to regenerate the loss of degenerated neurons and to provide a cure.

Another objective of the present invention is to improve the cognitive skills by improving neuro transmitters levels.

### SUMMARY OF THE INVENTION

The following summary is provided to facilitate a clear understanding of the new features in the disclosed embodiment and it is not intended to be a full, detailed description. A detailed description of all the aspects of the disclosed invention can be understood by reviewing the full specification, the drawing and the claims and the abstract, as a whole.

This present invention relates to a synergistic composition of phyto-active principles processed in a specific method (with enhanced therapeutic efficacy) to improve the cognitive skills (reliving the symptoms) by improving the levels of neurotransmitters. It also arrests the progressive degeneration of neuronal tissue in the brain and the central nervous system. This formulation is capable of preventing the formation of Beta Amyloid and Tau tangles. It also improves the circulation and enhances antioxidants levels in the brain and it has the potential to regenerate the neurons as well and therefore offers a cure in Alzheimer's disease and dementia and Parkinson's as well.

The present invention is the combination of phyto-active principles processed in lecithin, a Phospholipid. These ingredients undergo a special micro encapsulation process for better absorption and increased bio-availability and efficacy. Though the actual cause of Dementia and Alzheimer's Disease is not known, it is believed that, it can be due to ageing, free radical accumulation (due to slowing down of cellular antioxidant system), altered immunity which triggers Inflammatory chemokines, especially TNF alfa, and many other age-related and lifestyle and metabolic changes. One or the other or a combination of the above factors could lead to loss of mitochondrial membrane potential and mitochondrial degeneration, formation and accumulation of Amyloid Beta particles and Tau tangles which leads to neuro degeneration.

### BRIEF DESCRIPTION OF THE DRAWINGS

The manner in which the present invention is formulated is given a more particular description below, briefly summarized above, may be had by reference to the components, some of which is illustrated in the appended drawing It is to be noted; however, that the appended drawing illustrates only typical embodiments of this invention and are therefore should not be considered limiting of its scope, for the system may admit to other equally effective embodiments.

Throughout the drawings, the same drawing reference numerals will be understood to refer to the same elements and features.

The features and advantages of the present invention will become more apparent from the following detailed description a long with the accompanying figures, which forms a part of this application and in which:

FIG. 1: Block Diagram describing the components of our formulation in accordance with our present invention;

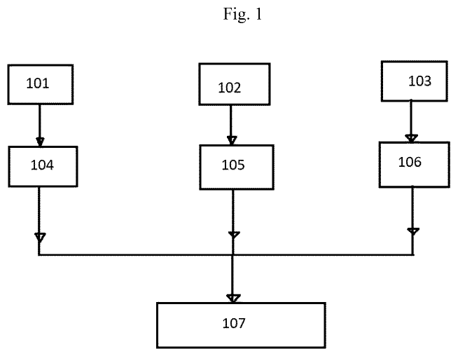

FIG. 2: Micro-pictographical representation describing the Histopathology Findings—Cresyl Violet Staining in accordance with our present invention;

### REFERENCE NUMERALS

101—Curcuminoid (Curcumin derivatives and analogues)
102—Andrographolides (Andrographolides derivatives and analogues)
103—Piperine
104—special process of lipid treatment and encapsulation
105—special lipid treatment and encapsulation with lecithin
106—special process of lipid treatment and encapsulation
107—phyto-active encapsulated formulation
201—Photomicrograph of Cresyl violet stained section (X100) of rat brain showing normal appearance of neuronal cells in the cerebral cortex region of brain (arrow) (Normal Group 1)
202—Photomicrograph of Cresyl violet stained section (X100) of rat brain showing normal appearance of neuronal cells in the hippocampal region of brain (arrow) (Normal Group 1)
203—Photomicrograph of Cresyl violet stained section (X100) of rat brain showing moderate neuronal cell dispersion in the cerebral cortex region of brain (arrow) (Diseased Group 2)
204—Photomicrograph of Cresyl violet stained section (X100) of rat brain showing High level neuronal cell dispersion in the hippocampal region of brain (arrow) (Diseased Group 2)
205—Photomicrograph of Cresyl violet stained section (X100) of rat brain showing normal appearance of neuronal cells in the cerebral cortex region of brain (arrow) (Treated Group 5)
206—Photomicrograph of Cresyl violet stained section (X100) of rat brain showing normal appearance of neuronal cells in the hippocampal region of brain (arrow) (Treated Group 5)

### DETAILED DESCRIPTION OF THE INVENTION

The principles of operation, design configurations and evaluation values in these non-limiting examples can be varied and are merely cited to illustrate at least one embodiment of the invention, without limiting the scope thereof.

The embodiments disclosed herein can be expressed in different forms and should not be considered as limited to the listed embodiments in the disclosed invention. The various embodiments outlined in the subsequent sections are constructed such that it provides a complete and a thorough understanding of the disclosed invention, by clearly describing the scope of the invention, for those skilled in the art.

Throughout this specification various indications have been given as to preferred and alternative embodiments of the invention. It should be understood that it is the appended claims, including all equivalents, which are intended to define the spirit and scope of this invention.

The present invention formulation works in synergy to improve the mitochondrial membrane potential and thereby prevent mitochondrial degeneration which is the root cause of neuro degenerative process. It also prevents the accumulation of free radicals by improving the cellular antioxidants levels and prevention of accumulation of Amyloid Beta particles and Tau Tangles formation. By preventing mitochondrial degeneration and by improving the mitochondrial health (improving Membrane potential) and cellular energy levels, it causes neuronal regeneration.

The present invention formulation is a combination of bioactive principles (Flavonoids) from plant source namely: Curcuminoids and Andrographolides.

Despite its range of reported pharmacological properties, they have poor bioavailability during oral administration and are poorly water soluble and therefore it is limited in its clinical applicability. Therefore, several strategies have been developed to overcome this disadvantage as, nanoparticles and formulations in liposomal, micellar phospholipid complex.

One of the most common strategies is to design and synthesize Curcumin derivatives and analogues. Our present formulation (107) contains all three Curcuminoids (101). This undergoes a special process of lipid treatment and encapsulation for better absorption and enhanced bio-availability for better therapeutic efficacy.

The formulation may contain either one of the curcuminoids or in combination or their analogues or their derivatives in other embodiments of the present invention.

The Second part of the formulation is: Andrographolide (102). Like Curcuminoids, Andrographolides are also lipophilic (Fat Soluble) phenolic compounds and they also exist along with three other derivatives, namely, 14-deoxy-11,12-dehydroandrographolide, Deoxy andrographolide and Neo andrographolide. Andrographolides are poorly water soluble and their absorption and bioavailability in biological system is poor.

The formulation may contain either one of the Andrographolides or in combination or their analogues or their derivatives in other embodiments of the present invention.

The present invention of formulation contains all three curcuminoids and all 4 andrographolides. Both curcuminoids and andrographolides, either one of Curcuminoids or in combination of them can be used in other embodiments of our present invention. Similarly, either all andrographolides or one of them or in combination of andrographolides can be used in another embodiment of our present invention. Piperine (103) or Black pepper can also be added in another embodiment of our present invention to increase the absorption and to increase its bio availability.

Like Curcuminoids (104), Andrographolides also undergo a special lipid treatment and encapsulation with lecithin (105) to enhance the absorption and to increase the bioavailability for better therapeutic results.

A cell line study conducted in IIT Madras to evaluate the efficacy and neuro regenerative properties of this compound (named PHY-001), in neurons against rotenone (Neuro toxin) induced neuro toxicity has proved its protective and regenerative properties through improving Mitochondrial health, Cellular Pyruvate and ATP levels and finally regenerative ability through MTT assay where maximum cell viability was noted with 100 micrograms of PHY-001 against 200 nM of rotenone (neurotoxin).

To elucidate the mechanism of action this compound was compared with a drug Terazosin, known for its ability to improve Pyruvate and ATP levels and to improve MMP (Mitochondrial Membrane Potential).

### In Vivo (Animal Study) at CEFTE of Sri Ramachandra Institute of Medical Education

This compound PHY-001 (Named as MAG XXI in Animal study by CEFTE) has also been subjected to in-vivo animal study at the animal study facility attached to SRMC (Sri Ramachandra Medical College) Hospital, Chennai. In this study, the therapeutic efficacy of this compound was evaluated against Alzheimer's Disease model, by inducing Alzheimer's like pathology by intra peritoneal administration of Aluminium Chloride (Established animal model for studying Alzheimer's disease and efficacy evaluation of a therapeutic intervention).

### COMPARATIVE STUDY

In a study the compound, MAG XXI was evaluated for its efficacy for relieving symptoms and correcting the underlying pathology.

The Behavioural studies like, Morris-Water Maze, Passive Avoidance Task and Elevated plus Maze were conducted. In these studies, our compound was comparatively evaluated against a presently available and widely prescribed (by Physicians worldwide) medication Donepezil.

Compared to Donepezil, this Phyto formulation has provided an equal and, in some cases, even better results in both behavioral as well as in histopathological studies in regulating the biomarkers levels.

More importantly as seen in the Cell line studies (MTT Assay, in IIT Madras) and in Histopathological studies (in CEFTE), this formulation has shown not only a protective effect against neuro toxins but also has shown to possess Neuro Regenerative ability and therefore capable of reversing the pathology.

Above all this formulation is totally devoid of any serious and undesirable side effects even at a dose of 400 mg per Kg body weight.

Based on the pilot study observations, a main study was conducted. Totally 30 Wistar male rats were used for the study. Animals were formed into five groups, Group 1-rats served as a control group, animals in group 2 to 5 were injected with AlCl3 intra-peritoneally once daily for four weeks. Before 1 hour of AlCl3 induction, Group 3 animals were administered orally with standard drug (Donepezil) and group 4 and 5 animals were administered orally with MAG XXI at 200 and 400 mg/Kg respectively for 28 days.

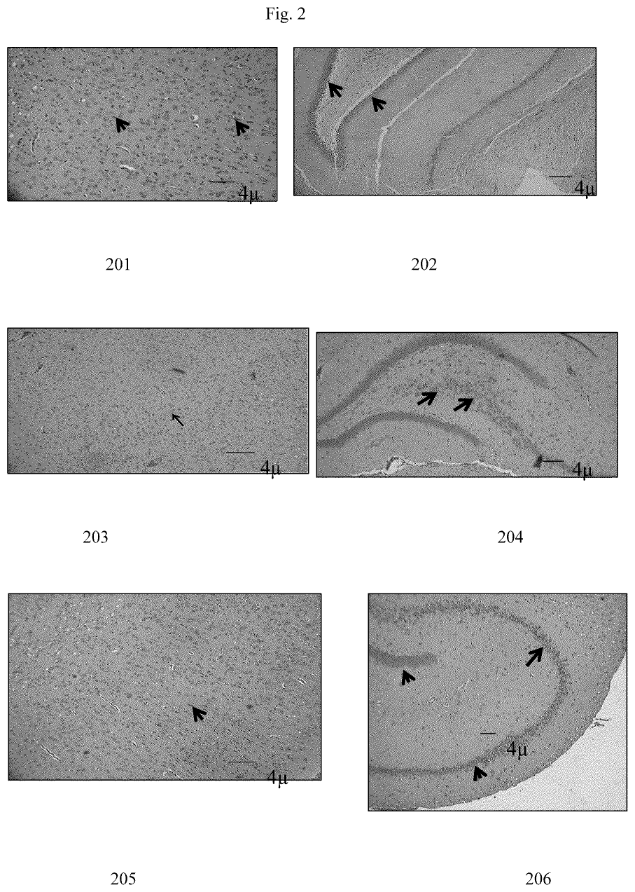

The results are depicted on FIG. 2, 201—Group 1 Photomicrograph of Cresyl violet stained section (X100) of rat brain showing normal appearance of neuronal cells in the cerebral cortex region of brain (arrow); 202—Group—Photomicrograph of Cresyl violet stained section (X100) of rat brain showing normal appearance of neuronal cells in the hippocampal region of brain (arrow); 203—Group 2—Photomicrograph of Cresyl violet stained section (X100) of rat brain showing moderate neuronal cell dispersion in the cerebral cortex region of brain (arrow); 204—Group 2—Photomicrograph of Cresyl violet stained section (X100) of rat brain showing High level neuronal cell dispersion in the hippocampal region of brain (arrow); 205—Photomicrograph of Cresyl violet stained section (X100) of rat brain showing normal appearance of neuronal cells in the cerebral cortex region of brain (arrow); 206—Photomicrograph of Cresyl violet stained section (X100) of rat brain showing normal appearance of neuronal cells in the hippocampal region of brain (arrow)

### Behaviour Test

The initial phase of AD (Alzheimer Disease) will result in short-term memory loss and progressively other disease signs like alterations in the mood and behaviour, aggression, confusion, avoiding people and social connections, and long-term memory loss. In the current study, through various behaviour tests, neuro degenerative effect of Aluminium Chloride was investigated.

### Morris Water Test

The results of number of times crossed centre at Morris water maze and escape latency which is the number of times the animal could find the platform and escape (in 3 minutes), are presented in table 1 & table 2.

Decrease in times crossed centre and escape latency at Morris water maze test at all weeks was noticed in G2 group which was significant at week 4 when compared to G1 which elucidate the neuronal pattern change especially on recognition and spatial memory. G3 and G5 showed significant elevation in times crossed centre and escape latency when compared to G2.

### Passive Avoidance Test

Significant alteration in dark compartment movement at all weeks is noticed in G2 group when compared to G1 which elucidate the learning memory defect. G3 and G5 show significant reversal when compared to G2.

### Elevated Plus Maze

The results of number of entries in open arm and closed arm at elevated plus mazes are presented in Table 4 and Table 5.

Decrease in number of entries in open arm and closed arm at all weeks is noticed in G2 group which is significant at week 4 when compared to G1 which elucidate the restricted movement due to feeling of insecurity. G3 and G5 showed significant elevation at week 4 when compared to G2.

TABLE 1
MORRIS WATER MAZE
No. OF TIMES CROSSED CENTER IN 1 MINUTE (MEAN VALUES)

| | WK 1 | WK 2 | WK 3 | WK 4 |
|---|---|---|---|---|
| G1 | 2.67 | 2.83 | 3 | 3.67 |
| G2 | 1.67 | 2 | 2.17 | 1.33 |
| G3 | 2.5 | 2.67 | 2.83 | 3.33 |
| G4 | 1.83 | 2.17 | 2.67 | 2.67 |
| G5 | 2.17 | 2.67 | 3.67 | 3.67 |

Result: G5 Could Achieve Normalcy (G1) In Week 3

TABLE 2
ESCAPE LATENCY (IN 3 MINUTES) No.
OF TIMES FIND P-LATFORM AND ESCAPE

| | WK 1 | WK 2 | WK 3 | WK 4 |
|---|---|---|---|---|
| G1 | 4.33 | 3.67 | 3.67 | 3.67 |
| G2 | 2.5 | 1.67 | 1.83 | 1.67 |
| G3 | 3.33 | 2.67 | 3.33 | 3.5 |
| G4 | 3.33 | 2.5 | 3.17 | 2.83 |
| G5 | 3.33 | 2.83 | 3.33 | 3.5 |

Result: G5 Could Show Better Than Other Groups

TABLE 3
PASSIVE AVOIDANCE (LATENCY TO ENTER
DARK CHAMBER-In Seconds)- MEAN VALUES

| | WK 1 | WK 2 | WK 3 | WK 4 |
|---|---|---|---|---|
| G1 | 40.17 | 43.67 | 46.67 | 46.67 |
| G2 | 15 | 16 | 16.17 | 14 |
| G3 | 31.83 | 42 | 45.33 | 47 |
| G4 | 21.5 | 24.67 | 24.33 | 27.17 |
| G5 | 36 | 39 | 42.83 | 46.83 |

TABLE 4
ELEVATED PLUS MAZE-No. OF ENTRIES in Open and Closed Arm

| | WK 1 | | WK 2 | | WK 3 | | WK 4 | |
|---|---|---|---|---|---|---|---|---|
| | Open | Closed | Open | Closed | Open | Closed | Open | Closed |
| G1 | 2.67 | 5.67 | 0.67 | 5.33 | 0.17 | 4 | 0.33 | 4.17 |
| G2 | 0 | 2 | 0.67 | 3.83 | 0 | 3.33 | 0.17 | 0.67 |
| G3 | 0.33 | 3.5 | 0.33 | 4.17 | 0 | 2.5 | 1.67 | 2.67 |
| G4 | 0 | 2.17 | 0 | 3 | 0 | 2.83 | 0.17 | 1.17 |
| G5 | 0.5 | 2.83 | 0.5 | 2.83 | 0.5 | 2.17 | 0.33 | 4.67 |

Result: G5 Could Perform Equal To G1 At 4th Week

TABLE 5
ELEVATED PLUS MAZE-Latency Period: 3 minutes
(UP = URINE POOL. DF = DEFACATION)

| | WK 1 | | WK2 | | WK 3 | | WK 4 | |
|---|---|---|---|---|---|---|---|---|
| | No. of | No. of | No. of | No. of | No. of | No. of | No. of | No. of |
| | UP | DF | UP | DF | UP | DF | UP | DF |
| G1 | 0.33 | 1.33 | 0.33 | 1 | 0.33 | 2.17 | 1.17 | 1.17 |
| G2 | 0.17 | 0.13 | 0.5 | 0.83 | 0.17 | 0.17 | 1.33 | 2.5 |
| G3 | 0.5 | 1 | 0.17 | 0.5 | 1.33 | 1.17 | 1.17 | 0.83 |
| G4 | 0.17 | 0.5 | 0.17 | 0 | 0.33 | 0.5 | 0.83 | 0.17 |
| G5 | 0.33 | 0.33 | 0.33 | 0 | 0.17 | 0.5 | 0 | 0.5 |

Results: G5 Are Much Better Compared To Other Groups

### Stress Marker—Antioxidant Level

Reactive Oxygen Species (ROS) are the prime players during the activation of numerous downstream signalling molecules like MAPKs. Reactive Oxygen Species resulting in oxidative stress may have a deleterious effect and can be an important mediator of damage to cell structures and consequently various disease states and aging. AD is also mediated through oxidative stress marker alterations, being a more complex disease due to the intervention of other makers. Frequent AlCl3 exposure imperatively increase free radicals (LPO-Lipid peroxide) and suppressed the antioxidants SOD (Super Oxide Dismutase), GTH (Glutathione) and GPx (Glutathione Peroxidase) status in the various brain portions that was supported in earlier reports. The present study results are presented in table.

In hippocampus regions, Aluminium Chloride induced animals, G2 showed significant increase in LPO with decrease in GPx, SOD and GSH levels compared on G1 (p<0.05 & 0.01). Whereas, oxidative stress and antioxidant markers were altered significantly decrease in G3, G4 and G5 (p<0.05 & 0.01) groups when compared to G2 group.

In cortex region, Aluminium Chloride induced animals, G2 showed significant increase in LPO with decrease in GPx, SOD and GSH levels compared on G1 (p<0.05). Whereas, reduced oxidative stress and significantly increased antioxidant markers were observed in G3 and G5 (p<0.05 & 0.01) groups when compared to G2 group.

These results prove that formulation found to be potent antioxidant at both tested doses (Tables 6 & Table 7)

### Inflammatory Markers

Neuroinflammation is the vital player of the initiation and progression of neuronal ailments. It could lead to memory and learning difficulties. The neuroinflammation has the crucial role in the pathological progression of the neuronal ailments like AD. Accumulating evidence has suggested that inflammatory responses play a critical role in the neurodegenerative cascades of AD and several markers have some tracing and detecting accuracy for disease severity and progression. Inflammatory markers such as the tumour necrosis factor α (TNF-α), and Interleukin-6 (IL-6) have been reported as important signalling molecules in inflammation that exert effects on the brains of people with dementia. The results of inflammatory markers measured in brain tissue was presented in Tables 8 & Table 9.

In hippocampus and cortex regions, Aluminium Chloride induced animals (G2) showed significant increase in TNF-α, IL-6 and NF_{K}B compared to G1 (p>0.05 and p<0.01). However, when compared to G2 group, inflammatory markers levels were statistically significantly decreased from G3, G4 and G5 (p<0.05 and p<0.01).

### Neuronal Markers

Results of neuronal markers are presented in Tables 10 & Table 11.

Also, it was exhibited in our present study, where AlCl3-triggered animals of G2notably increase in the AChE content, Beta Amyloid, Gamma secretase, Beta secretase, APP levels mainly in hippocampal region when compared to cortex. However, neuronal markers levels were brought down in G3, G4 and statistically significantly reduced in G5 (p<0.05 and 0.01) compared to G2 at both brain regions.

TABLE 6
OXIDATIVE MARKERS IN HIPPOCAMPAL
TISSUE - MEAN VALUES

| | LPO | SOD | GSH | GPX |
|---|---|---|---|---|
| G 1 | 0.59 | 0.49 | 14.45 | 11.8 |
| G 2 | 0.97 | 0.24 | 8.13 | 7.86 |
| G 3 | 0.81 | 0.26 | 8.27 | 9.43 |
| G 4 | 0.82 | 0.58 | 12.2 | 14.99 |
| G 5 | 0.81 | 0.6 | 12.58 | 16.27 |

RESULT: Significant Decrease in Free radical LPO and increase in Antioxidant levels in G5 Vs G2 (Alcl) & G3 (Donepezil)

TABLE 7
OXIDATIVE MARKERS IN CORTEX
TISSUE - MEAN VALUES

| | LPO | SOD | GSH | GPX |
|---|---|---|---|---|
| G1 | 1.35 | 0.59 | 32.85 | 18.19 |
| G2 | 2.08 | 0.37 | 24.51 | 9.67 |
| G3 | 1.72 | 0.46 | 26.33 | 7.42 |
| G4 | 1.78 | 0.61 | 29.58 | 13.32 |
| G5 | 1.62 | 0.64 | 31.13 | 14.72 |

Result: Significant decrease in LPO (Free Radical) and increase in Anti-oxidants levels in G5 Vs G2 (Alcl) & G3 (Donepezil)

TABLE 8
INFLAMMATORY MARKERS IN HIPPOCAMPUS
TISSUE -MEAN VALUES

| | IL-6 | TNFα | NFkB |
|---|---|---|---|
| G1 | 1741.5 | 18.73 | 0.88 |
| G2 | 2233.33 | 30.48 | 1.56 |
| G3 | 1676.83 | 24.06 | 1.06 |
| G4 | 1662.67 | 22.57 | 1.05 |
| G5 | 1495.5 | 22.15 | 0.96 |

Result: Significant Reduction in inflammatory markers in G5 Vs G2 & G3

TABLE 9
INFLAMATORY MARKERS IN CORTEX
TISSUE - MEAN VALUES

| | IL-6 | TNFα | NFkB |
|---|---|---|---|
| G1 | 1424.33 | 21.87 | 1.14 |
| G2 | 2509.33 | 31.82 | 1.68 |
| G3 | 1692.33 | 19.55 | 1.22 |
| G4 | 1686.67 | 22.33 | 1.3 |
| G5 | 1465.33 | 22.64 | 1.01 |

Result: Significant Reduction in inflammatory markers in G5 Vs G2 & G3

TABLE 10
NURONAL MARKERS IN HIPPOCAMPUS - MEAN VALUES

| | B | | B | |
|---|---|---|---|---|
| | Amyloid | γ Secretase | Secretase | APP |
| G1 | 230.61 | 1053.53 | 7.9 | 510.64 |
| G2 | 368.82 | 1430.01 | 10.09 | 1063.36 |
| G3 | 257.9 | 1110.57 | 7.57 | 471.17 |
| G4 | 234.35 | 1219.78 | 8.14 | 476.92 |
| G5 | 225.93 | 1063.67 | 7.4 | 444.67 |

Result: Significant Reduction in values in G5 Vs G2 & G3

TABLE 11
NEURONAL MARKERS IN CORTEX - MEAN VALUES

| | B | γ | B | |
|---|---|---|---|---|
| | Amyloid | Secretase | APP | Secretase |
| G1 | 308.04 | 990.46 | 4.83 | 608.08 |
| G2 | 441.83 | 1361.43 | 7.93 | 1061.75 |
| G3 | 328.72 | 869.29 | 6.54 | 611.78 |
| G4 | 355.44 | 1069.52 | 6.6 | 768.58 |
| G5 | 318.89 | 1053.6 | 5.9 | 660.42 |

Result: Significant Reduction in values in G5 Vs G2 and G3

### Gross Pathology

No gross lesions were observed in all animals of five groups during the necropsy.

### Histopathology

Representative images of Histopathology Findings—Cresyl Violet Staining are displayed in FIG. 2.
No histopathological findings were observed in all animals of Group 1.
In the Group 2, histopathology of brain revealed minimal to mild severity of NFTs in hippocampus (06/06) and cerebral cortex (06/06). Gliosis was observed in 03 out of 06 animals with minimal (01/06) to mild (02/06) severity. Mild severity of glial nodule and mononuclear cell infiltration were observed in 01 out of 06 animals. Minimal (01/06) to Mild (01/06) degree of Neuronal degeneration was found in 02 out of 06 animals.
In the Group 3, histopathology of brain revealed minimal to mild severity of NFTs in hippocampus (05/06) and cerebral cortex (06/06). Gliosis was observed in 02 out of 06 animals with minimal (01/06) to mild (01/06) severity. Neuronal degeneration, Glial nodule and mononuclear cell infiltration were not observed in any animals of this group.
In the Group 4, histopathology of brain revealed minimal to mild severity of NFTs in hippocampus (05/06) and cerebral cortex (06/06). Gliosis was observed in 02 out of 06 animals with mild (02/06) severity. Minimal severity of mononuclear cell infiltration was observed in 01 out of 06 animals. Neuronal degeneration and Glial nodule were not observed in any animals of this group.
In the Group 5, histopathology of brain revealed minimal to mild severity of NFTs in hippocampus (04/06) and cerebral cortex (06/06). Gliosis, Neuronal degeneration, Glial nodule and mononuclear cell infiltration were not observed in any animals of this group. Incidence of histopathological findings was found less in G5 when compared with G2 & G4.

Also, it was exhibited in our present study, where AlCl3-triggered animals G2 notably increased in the AChE content, Beta Amyloid, Gamma secretase, Beta secretase, APP levels mainly in hippocampal region when compared to cortex. However, neuronal markers levels were statistically significantly reduced and it is near to normal in G3, G4 and G5 (p<0.05 and 0.01).

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope of the invention as claimed.

I/We claim:

1. A phytoactive encapsulated formulation preparation which consists of a phospholipid encapsulated formulation of curcuminoids and andrographolides, wherein,
a. the formulation can contain either one of the curcuminoids (101) or in combination or their analogues or their derivatives;
b. the formulation can contain either one of the andrographolides (102) or in combination or their analogues or their derivatives; and
c. the formulation (107) may contain either one of the curcuminoids or andrographolides or a combination of curcuminoids and andrographolides as represented by claim 1(a) and 1(b).

2. The phytoactive encapsulated formulation preparation, as claimed in claim 1, wherein, both the compounds undergo a special process of lipid treatment and encapsulation preferably with lecithin (104, 105).

3. The phytoactive encapsulated formulation preparation, as claimed in claim 1, wherein, Piperine (103) (Extracted from Black pepper) or Black pepper itself can also be added to the formulation.
